(19) United States
(12) Patent Application Publication
(10) Pub. No.: US 2022/0248733 A1
(43) Pub. Date: Aug. 11, 2022

(54) FORMULATIONS INCLUDING DIHYDROHONOKIOL

(71) Applicant: Justin KIRKLAND, Champaign, IL (US)

(72) Inventor: Justin KIRKLAND, Champaign, IL (US)

(21) Appl. No.: 17/666,407

(22) Filed: Feb. 7, 2022

Related U.S. Application Data
(63) Continuation of application No. PCT/US20/45089, filed on Aug. 6, 2020.
(60) Provisional application No. 62/883,453, filed on Aug. 6, 2019.

(51) Int. Cl. A23L 33/105; A23C 21/08

(52) U.S. Cl. CPC A23L 33/105; A23C 2240/15; A23C 21/08

(57) ABSTRACT

Disclosed herein are compositions for treating anxiety and related disorders. In some embodiments, the compositions comprise dihydrohonokiol-B (“DHH-B”) and at least one pharmaceutically acceptable carrier, wherein the DHH-B is present in an amount ranging from between about 0.5% to about 25% by total weight of the composition. In other embodiments, the compositions comprise dihydrohonokiol-B (“DHH-B”) and at least one pharmaceutically acceptable carrier, wherein the DHH-B is present in an amount ranging from between about 0.000001% to about 5% by total weight of the composition.

### CROSS-REFERENCE TO RELATED APPLICATIONS

The present disclosure is a continuation of International Application No. PCT/US20/45089 filed on Aug. 6, 2020, which application claims the benefit of the filing date of U.S. Patent Application No. 62/883,453, filed on Aug. 6, 2019, the disclosure of which is hereby incorporated by reference herein in their entireties.

### BACKGROUND OF THE DISCLOSURE

Anxiety disorders have heretofore been considered to be types of nervous diseases. Examples of representative symptoms of anxiety disorders include nervous disorders, mood disorders, personality disorders, behavior disorders, and sleep disorders. Approximately 50 products, such as benzodiazepines, thienodiazepines, and carbamate preparations, are known as pharmaceutical preparations used for treatment of the symptoms mentioned above.

Since long-term administration of such pharmaceutical preparations is required in order to improve symptoms, disadvantageously, use of such pharmaceutical preparations can cause severe side effects, such as drug dependence, motility disorders, and confusion, or minor side effects, such as drowsiness, dizziness, loss of appetite, and weakness. Accordingly, development of a novel anti-anxiety formulation that can serve as an alternative to existing anti-anxiety agents has been awaited.

### BRIEF SUMMARY OF THE DISCLOSURE

In one aspect of the present disclosure are formulations comprising dihydrohonokiol-B (DHH-B) or a derivative or analog thereof (hereinafter the “DHH-B formulation”). In some embodiments, the DHH-B formulations may be in the form of a paste, a powder, an oil, a liquid, a suspension, a solution, or other form. In some embodiments, the DHH-B formulations suitable for administration to a mammal. In some embodiments, the DHH-B formulations are provided for administration to a human subject. In other embodiments, the DHH-B formulations are provided for veterinary use. In some embodiments, the DHH-B formulations are suitable for treating anxiety or an anxiety-related disorder.

In some embodiments, the DHH-B formulations are provided as a liquid formulation for oral administration, such as for oral administration to a human and/or veterinary subject. In some embodiments, liquid formulations including DHH-B may take the form of, for example, solutions, syrups or suspensions, or they may be presented as a dry product for reconstitution with water or another suitable vehicle prior to administration and may be administered to human and/or veterinary subjects. Such liquid formulations may be prepared by conventional means with pharmaceutically acceptable additives such as suspending agents (e.g., sorbitol syrup, methyl cellulose, or hydrogenated edible fats); emulsifying agents (e.g., lecithin or acacia); non-aqueous vehicles (e.g., almond oil, oily esters or ethyl alcohol); preservatives (e.g., methyl or propyl p-hydroxybenzoates or sorbic acid); binders, disintegrants, fillers, complexing agents, lubricants, and/or artificial or natural colors or sweeteners. Liquid formulations can be administered to humans or veterinary subjects in pharmaceutical carriers known to those skilled in the art. Such pharmaceutical carriers include, but are not limited to, capsules, lozenges, syrups, sprays, rinses, and mouthwash.

In some embodiments, the DHH-B formulations may be utilized as dietary supplements, nutraceuticals, or such other preparations that may be used to prevent, mitigate, slow the onset of, or treat various human ailments, e.g. anxiety. It will be recognized that dietary supplements including DHH-B may not use the same formulation ingredients or have the same sterile and other FDA requirements as pharmaceutical compositions. The dietary supplements may be in liquid form, for example, solutions, syrups or suspensions, or may be in the form of a product for reconstitution with water or any other suitable liquid before use. Such liquid preparations may be prepared by conventional means such as a tea, health beverage, dietary shake, liquid concentrate, or liquid soluble tablet, capsule, pill, or powder such that the beverage may be prepared by dissolving the liquid soluble tablet, capsule, pill, or powder within a liquid and consuming the resulting beverage. Alternatively, the dietary supplements including DHH-B may take the form of tablets or capsules, such as soft gel capsules, prepared by conventional means and optionally including other dietary supplements including vitamins, minerals, other herbal supplements, binding agents, fillers, lubricants, disintegrants, or wetting agents, as those discussed above. The tablets may be coated by methods well-known in the art to provide for delayed release and/or extended release.

A first aspect of the present disclosure is a composition comprising dihydrohonokiol-B (“DHH-B”) and at least one pharmaceutically acceptable carrier, wherein the DHH-B is present in an amount ranging from between about 0.5% to about 25% by total weight of the composition. In some embodiments, the DHH-B is complexed with a solubilizer. In some embodiments, the solubilizer is a cyclodextrin. In some embodiments, the cyclodextrin is hydroxypropyl-β-cyclodextrin. In some embodiments, the composition further comprises at least three of a diluent, a binder, a sweetener, a disintegrant, a filler, and a lubricant. In some embodiments, the composition further comprises crospovidone in an amount ranging from between about 0.4% to about 65% by total weight of the composition.

In some embodiments, the composition comprises microcrystalline cellulose in an amount ranging from between about 39% to about 80% by total weight of the composition. In some embodiments, the DHH-B is present in an amount ranging from between about 0.75% to about 2.5% by total weight of the composition.

In some embodiments, the composition further comprises an oil and a surfactant. In some embodiments, the oil comprises a triglyceride. In some embodiments, the surfactant comprises polyethyleneglycol having an average molecular weight of about 1500 g/mol. In some embodiments, the composition further comprises diethylene glycol monoethyl ether.

A second aspect of the present disclosure is a composition comprising dihydrohonokiol-B (“DHH-B”) and at least one pharmaceutically acceptable carrier, wherein the DHH-B is present in an amount ranging from between about 0.000001% to about 5% by total weight of the composition. The composition of claim 13, further comprising D-α-tocopheryl polyethylene glycol succinate. In some embodiments, the composition further comprises a flavoring agent. In some embodiments, the DHH-B is present in an amount ranging from between about 0.000008% to about 1% by total weight of the composition.

In some embodiments, the composition further comprises a wetting agent. In some embodiments, the wetting agent is propylene glycol, and wherein the propylene glycol is present in an amount ranging from between about 0.0005% to about 6% by total weight of the composition.

A third aspect of the present disclosure is a method of treating anxiety, or the symptoms thereof, comprising administering to a subject in need thereof a composition comprising dihydrohonokiol-B (“DHH-B”) and at least one pharmaceutically acceptable carrier, wherein the DHH-B is present in an amount ranging from between about 0.5% to about 25% by total weight of the composition. In some embodiments, the formulation is administered such that at least 5mg of the DHH-B is administered to the subject per day. In some embodiments, the method further comprises co-administering a second active pharmaceutical ingredient to the subject, wherein the second active pharmaceutical ingredient is an anxiolytic agent.

A fourth aspect of the present disclosure is a method of treating anxiety, or the symptoms thereof, comprising administering to a subject in need thereof a composition comprising dihydrohonokiol-B (“DHH-B”) and at least one pharmaceutically acceptable carrier, wherein the DHH-B is present in an amount ranging from between about 0.000001% to about 5% by total weight of the composition.

A fifth aspect of the present disclosure is a method of treating essential tremor disorder, Parkinsonian tremor, dystonic tremor, cerebellar tremor, psychogenic tremor, orthostatic tremor, physiologic tremor comprising administering to a subject in need thereof a composition comprising dihydrohonokiol-B (“DHH-B”) and at least one pharmaceutically acceptable carrier, wherein the DHH-B is present in an amount ranging from between about 0.000001% to about 5% by total weight of the composition.

A sixth aspect of the present disclosure is a method of treating essential tremor disorder, Parkinsonian tremor, dystonic tremor, cerebellar tremor, psychogenic tremor, orthostatic tremor, physiologic tremor comprising administering to a subject in need thereof a composition comprising dihydrohonokiol-B (“DHH-B”) and at least one pharmaceutically acceptable carrier, wherein the DHH-B is present in an amount ranging from between about 0.5% to about 25% by total weight of the composition. In some embodiments, the formulation is administered such that at least 5 mg of the DHH-B is administered to the subject per day.

### DETAILED DESCRIPTION

### Definitions

It should also be understood that, unless clearly indicated to the contrary, in any methods claimed herein that include more than one step or act, the order of the steps or acts of the method is not necessarily limited to the order in which the steps or acts of the method are recited.

As used herein, the singular terms “a,” “an,” and “the” include plural referents unless context clearly indicates otherwise. Similarly, the word “or” is intended to include “and” unless the context clearly indicates otherwise. The term “includes” is defined inclusively, such that “includes A or B” means including A, B, or A and B.

As used herein, the term “about” is used herein to mean approximately, in the region of, roughly, or around. When the term “about” is used in conjunction with a numerical range, it modifies that range by extending the boundaries above and below the numerical values set forth. In general, the term “about” or “approximately” is used herein to modify a numerical value above and below the stated value by a variance of 20%.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of” or “exactly one of.” “Consisting essentially of,” when used in the claims, shall have its ordinary meaning as used in the field of patent law.

The terms “comprising,” “including,” “having,” and the like are used interchangeably and have the same meaning. Similarly, “comprises,” “includes,” “has,” and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The term “anti-anxiety action” or “anti-anxiety effect” used herein refers to, for example, treatment, alleviation, or prevention of anxiety disorder conditions, such as nervous disorders, mood disorders, personality disorders, behavior disorders, and sleep disorders.

As used herein, the term “administering” means providing a composition, formulation, or specific agent to a subject in need of treatment, including those described herein.

The phrases “pharmaceutically acceptable” or “pharmacologically acceptable” refer to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the expression vectors of the present disclosure, its use in therapeutic compositions is contemplated.

As used herein, the term “subject” refers to a mammal such as a human, mouse, a horse, a dog, or a primate. Typically, the mammal is a human (homo sapiens). A human subject may be an adult patient or a pediatric patient.

As used herein, the terms “therapeutically effective dose” or “dose amount” refer to an amount of a composition, or a component of the composition, which is effective to achieve an improvement in a subject or his physiological systems including, but not limited to, improved improvement or elimination of symptoms, delayed onset of a disorder, slower progress of symptoms and other indicators selected as appropriate by those skilled in the art.

As used herein, the terms “treatment,” “treating,” or “treat,” with respect to a specific condition, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease in a subject, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease and/or relieving one or more disease symptoms. “Treatment” can also encompass delivery of an agent or administration of a therapy in order to provide for a pharmacologic effect, even in the absence of a disease or condition. The term “treatment” is used in some embodiments to refer to administration of a compound of the present disclosure to mitigate a disease or a disorder in a host, preferably in a mammalian subject, more preferably in humans. Thus, the term “treatment” can include includes: preventing a disorder from occurring in a host, particularly when the host is predisposed to acquiring the disease but has not yet been diagnosed with the disease; inhibiting the disorder; and/or alleviating or reversing the disorder. Insofar as the methods of the present disclosure are directed to preventing disorders, it is understood that the term “prevent” does not require that the disease state be completely thwarted. Rather, as used herein, the term preventing refers to the ability of the skilled artisan to identify a population that is susceptible to disorders, such that administration of the compounds of the present disclosure can occur prior to onset of a disease. The term does not mean that the disease state must be completely avoided.

### DHH-B Formulations

The present disclosure is directed to DHH-B formulations suitable for administration to a mammal. In some embodiments, the DHH-B formulations are provided for administration to a human subject. In other embodiments, the DHH-B formulations are provided for veterinary use.

### Dihydrohonokiol-B

Dihydrohonokiol, 3′-(2-propenyl)-5-propyl-(1,1′-biphenyl)-2,4′-diol (“DHH-B”), is a potent anxiolytic compound, developed benzodiazepine-like side effects.

In some embodiments, an amount of DHH-B within any formulation ranges from between about 0.5% to about 30% by total weight of the composition. In other embodiments, an amount of DHH-B within any composition ranges from between about 1% to about 30% by total weight of the composition. In yet other embodiments, an amount of DHH-B within any composition ranges from between about 1% to about 25% by total weight of the composition. In further embodiments, an amount of DHH-B within any composition ranges from between about 1% to about 20% by total weight of the composition. In even further embodiments, an amount of DHH-B within any composition ranges from between about 1% to about 15% by total weight of the composition. In yet further embodiments, an amount of DHH-B within any composition ranges from between about 1% to about 10% by total weight of the composition. In yet even further embodiments, an amount of DHH-B within any composition ranges from between about 1% to about 5% by total weight of the composition.

In some embodiments, an amount of DHH-B within any formulation ranges from between about 0.000001% to about 5% by total weight of the composition. In other embodiments, an amount of DHH-B within any composition ranges from between about 0.000001% to about 3% by total weight of the composition. In yet other embodiments, an amount of DHH-B within any composition ranges from between about 0.000001% to about 2.5% by total weight of the composition. In further embodiments, an amount of DHH-B within any composition ranges from between about 0.00001% to about 2.5% by total weight of the composition. In even further embodiments, an amount of DHH-B within any composition ranges from between about 0.0001% to about 2.5% by total weight of the composition. In yet further embodiments, an amount of DHH-B within any composition ranges from between about 0.001% to about 2.5% by total weight of the composition. In yet even further embodiments, an amount of DHH-B within any composition ranges from between about 0.01% to about 2.5% by total weight of the composition. In yet even further embodiments, an amount of DHH-B within any composition ranges from between about 0.01% to about 2% by total weight of the composition. In yet even further embodiments, an amount of DHH-B within any composition ranges from between about 0.01% to about 1.5% by total weight of the composition. In yet even further embodiments, an amount of DHH-B within any composition ranges from between about 0.1% to about 1.5% by total weight of the composition. In yet even further embodiments, an amount of DHH-B within any composition ranges from between about 0.1% to about 1% by total weight of the composition. In yet even further embodiments, an amount of DHH-B within any composition ranges from between about 0.1% to about 0.5% by total weight of the composition.

In some embodiments, an amount of DHH-B within any formulation ranges from between about 1 mg to about 20 mg of DHH-B. In some embodiments, an amount of DHH-B within any formulation ranges from between about 2 mg to about 20 mg of DHH-B. In some embodiments, an amount of DHH-B within any formulation ranges from between about 3 mg to about 18 mg of DHH-B. In some embodiments, an amount of DHH-B within any formulation ranges from between about 3 mg to about 16 mg of DHH-B. In some embodiments, an amount of DHH-B within any formulation ranges from between about 3 mg to about 15 mg of DHH-B. In some embodiments, an amount of DHH-B within any formulation ranges from between about 4 mg to about 14 mg of DHH-B. In some embodiments, an amount of DHH-B within any formulation ranges from between about 4 mg to about 12 mg of DHH-B. In some embodiments, an amount of DHH-B within any formulation ranges from between about 5 mg to about 10 mg of DHH-B.

In some embodiments, an amount of DHH-B within any formulation is about 4 mg. In some embodiments, an amount of DHH-B within any formulation is about 4.5 mg. In some embodiments, an amount of DHH-B within any formulation is about 5 mg. In some embodiments, an amount of DHH-B within any formulation is about 5.5 mg. In some embodiments, an amount of DHH-B within any formulation is about 6 mg. In some embodiments, an amount of DHH-B within any formulation is about 6.5 mg. In some embodiments, an amount of DHH-B within any formulation is about 7 mg. In some embodiments, an amount of DHH-B within any formulation is about 7.5 mg. In some embodiments, an amount of DHH-B within any formulation is about 8 mg. In some embodiments, an amount of DHH-B within any formulation is about 8.5 mg. In some embodiments, an amount of DHH-B within any formulation is about 9 mg. In some embodiments, an amount of DHH-B within any formulation is about 9.5 mg. In some embodiments, an amount of DHH-B within any formulation is about 10 mg. In some embodiments, an amount of DHH-B within any formulation is about 10.5 mg. In some embodiments, an amount of DHH-B within any formulation is about 11 mg. In some embodiments, an amount of DHH-B within any formulation is about 11.5 mg. In some embodiments, an amount of DHH-B within any formulation is about 12 mg. In some embodiments, an amount of DHH-B within any formulation is about 12.5 mg. In some embodiments, an amount of DHH-B within any formulation is about 13 mg. In some embodiments, an amount of DHH-B within any formulation is about 15 mg.

In some embodiments, the daily dose of DHH-B ranges from between about 0.08 mg/kg to about 3 mg/kg. In some embodiments, the daily dose of DHH-B ranges from between about 0.09 mg/kg to 2.8 mg/kg. In some embodiments, the daily dose of DHH-B ranges from between about 0.1 mg/kg to about 2.6 mg/kg. In some embodiments, the daily dose of DHH-B ranges from between about 0.11 mg/kg to about 2.4 mg/kg. In some embodiments, the daily dose of DHH-B ranges from between about 0.12 mg/kg to about 2.2 mg/kg. In some embodiments, the daily dose of DHH-B ranges from between about 0.13 mg/kg to about 2 mg/kg. In some embodiments, the daily dose of DHH-B ranges from between about 0.14 mg/kg to about 1.8 mg/kg. In some embodiments, the daily dose of DHH-B is about 0.15 mg/kg.

### Pharmaceutically Acceptable Excipients, Carriers, and Additives

The formulations of the present disclosure may further comprise one or more pharmaceutically acceptable excipients including, but not limited to, diluents, binders, lubricants, disintegrants, flavoring agents, taste-masking agents, coloring agents, pH modifiers, stabilizers, absorption enhancers, viscosity modifiers, film forming polymers, bulking agents, surfactants, glidants, plasticizers, preservatives, essential oils and sweeteners. In some embodiments, the pharmaceutically acceptable excipients, carriers, and/or additives may be a food composition or a food product into which formulations described herein may be introduced.

A person skilled in the art will be able to select the suitable excipients or mixtures of excipients for the desired formulations. In general, the amount of any pharmaceutically acceptable excipient, carrier, and/or additive included within any formulations may vary depending on the desired effect, route of administration, form of the final composition. In general, however, a total amount of pharmaceutically acceptable excipients, carriers, and/or additives formulated with the formulations of the present disclosure may range from about 1% to about 99% by total weight of the formulations. In some embodiments, a total amount of pharmaceutically acceptable excipients, carriers, and/or additives formulated with the formulations of the present disclosure may range from about 1% to about 98% by total weight of the formulations. In some embodiments, a total amount of pharmaceutically acceptable excipients, carriers, and/or additives formulated with the formulations of the present disclosure may range from about 1% to about 97% by total weight of the formulations. In some embodiments, a total amount of pharmaceutically acceptable excipients, carriers, and/or additives formulated with the formulations of the present disclosure may range from about 1% to about 96% by total weight of the formulations. In some embodiments, a total amount of pharmaceutically acceptable excipients, carriers, and/or additives formulated with the formulations of the present disclosure may range from about 1% to about 95% by total weight of the formulations. In some embodiments, a total amount of pharmaceutically acceptable excipients, carriers, and/or additives formulated with the formulations of the present disclosure may range from about 1% to about 94% by total weight of the formulations. In some embodiments, a total amount of pharmaceutically acceptable excipients, carriers, and/or additives formulated with the formulations of the present disclosure may range from about 1% to about 93% by total weight of the formulations. In some embodiments, a total amount of pharmaceutically acceptable excipients, carriers, and/or additives formulated with the formulations of the present disclosure may range from about 1% to about 92% by total weight of the formulations. In some embodiments, a total amount of pharmaceutically acceptable excipients, carriers, and/or additives formulated with the formulations of the present disclosure may range from about 1% to about 91% by total weight of the formulations.

In other embodiments, the total amount of pharmaceutically acceptable excipients, carriers, and/or additives formulated with the formulations of the present disclosure may range from about 1% to about 90% by total weight of the formulation. In some embodiments, a total amount of pharmaceutically acceptable excipients, carriers, and/or additives formulated with the formulations of the present disclosure may range from about 1% to about 88% by total weight of the formulations. In some embodiments, a total amount of pharmaceutically acceptable excipients, carriers, and/or additives formulated with the formulations of the present disclosure may range from about 1% to about 86% by total weight of the formulations. In some embodiments, a total amount of pharmaceutically acceptable excipients, carriers, and/or additives formulated with the formulations of the present disclosure may range from about 1% to about 84% by total weight of the formulations. In some embodiments, a total amount of pharmaceutically acceptable excipients, carriers, and/or additives formulated with the formulations of the present disclosure may range from about 1% to about 82% by total weight of the formulations. In other embodiments, the total amount of pharmaceutically acceptable excipients, carriers, and/or additives formulated with the formulations of the present disclosure may range from about 1% to about 80% by total weight of the formulation. In some embodiments, a total amount of pharmaceutically acceptable excipients, carriers, and/or additives formulated with the formulations of the present disclosure may range from about 1% to about 78% by total weight of the formulations. In some embodiments, a total amount of pharmaceutically acceptable excipients, carriers, and/or additives formulated with the formulations of the present disclosure may range from about 1% to about 76% by total weight of the formulations. In some embodiments, a total amount of pharmaceutically acceptable excipients, carriers, and/or additives formulated with the formulations of the present disclosure may range from about 1% to about 74% by total weight of the formulations. In some embodiments, a total amount of pharmaceutically acceptable excipients, carriers, and/or additives formulated with the formulations of the present disclosure may range from about 1% to about 72% by total weight of the formulations. In other embodiments, the total amount of pharmaceutically acceptable excipients, carriers, and/or additives formulated with the formulations of the present disclosure may range from about 1% to about 70% by total weight of the formulation.

In some embodiments, a ratio of an amount of DHH-B an amount of a pharmaceutically acceptable excipient or carrier ranges from between about 100:1 to about 1:100. In some embodiments, a ratio of an amount of DHH-B an amount of a pharmaceutically acceptable excipient or carrier ranges from between about 90:1 to about 1:90. In some embodiments, a ratio of an amount of DHH-B an amount of a pharmaceutically acceptable excipient or carrier ranges from between about 80:1 to about 1:80. In some embodiments, a ratio of an amount of DHH-B an amount of a pharmaceutically acceptable excipient or carrier ranges from between about 70:1 to about 1:70. In some embodiments, a ratio of an amount of DHH-B an amount of a pharmaceutically acceptable excipient or carrier ranges from between about 60:1 to about 1:60. In some embodiments, a ratio of an amount of DHH-B and an amount of a pharmaceutically acceptable excipient or carrier ranges from about 50:1 to about 1:50. In some embodiments, a ratio of an amount of DHH-B an amount of a pharmaceutically acceptable excipient or carrier ranges from between about 40:1 to about 1:40. In some embodiments, a ratio of an amount of DHH-B and an amount of a pharmaceutically acceptable excipient or carrier ranges from about 30:1 to about 1:30. In some embodiments, a ratio of an amount of DHH-B an amount of a pharmaceutically acceptable excipient or carrier ranges from between about 20:1 to about 1:20. In some embodiments, a ratio of an amount of DHH-B and an amount of a pharmaceutically acceptable excipient or carrier ranges from about 10:1 to about 1:10. In some embodiments, a ratio of an amount of DHH-B and an amount of a pharmaceutically acceptable excipient or carrier ranges from about 5:1 to about 1:5.

### Water

In some embodiments, the carrier is water. In some embodiments, an amount of water present in the composition ranges from about 80% to about 99% by total weight of the composition, from about 80% to about 98% by total weight of the composition, from about 85% to about 97% by total weight of the composition, from about 85% to about 95% by total weight of the composition, from about 88% to about 95% by total weight of the composition, from about 90% to about 95% by total weight of the composition, from about 90% to about 94% by total weight of the composition, and from about 90% to about 93% by total weight of the composition. By way of example only, a formulation may comprise a 50:50 mixture of an active agent (e.g. DHH-B) and a pharmaceutically acceptable excipient, carrier, and/or additive.

### Diluents

A diluent may be selected from, for example, calcium carbonate, calcium phosphate dibasic, calcium phosphate tribasic, calcium sulfate, microcrystalline cellulose, microcrystalline silicified cellulose, powdered cellulose, dextrate, dextrose, fructose, lactitol, lactose anhydrous, lactose monohydrate, lactose dihydrate, lactose trihydrate, mannitol, sorbitol, starch, pregelatinized starch, sucrose, talc, xylitol, maltose, maltodextrin, maltitol. In some embodiments, the diluent is selected from starches, lactose, cellulose derivatives, confectioner's sugar and the like. Different grades of lactose include, but are not limited to, lactose monohydrate, lactose DT (direct tableting), lactose anhydrous, and others. Different starches include, but are not limited to, maize starch, potato starch, rice starch, wheat starch, pregelatinized starch, and others . Different celluloses that can be used include crystalline celluloses, such as a microcrystalline cellulose, and powdered celluloses. Other useful diluents include, but are not limited to, carmellose, sugar alcohols such as mannitol, sorbitol, and xylitol, calcium carbonate, magnesium carbonate, dibasic calcium phosphate, and tribasic calcium phosphate.

### Binders

A binder may be selected from, for example, acacia, alginic acid, carbomer, carboxymethylcellulose calcium, carbomethylcellulose sodium, microcrystalline cellulose, powdered cellulose, ethyl cellulose, gelatin liquid glucose, guar gum, hydroxyethyl cellulose, hydroxypropyl cellulose, hydroxypropylmethyl cellulose, maltodextrin, methylcellulose, polydextrose, polyethtylene oxide, povidone, sodium alginate, starch paste, pregelatinized starch, sucrose, tragacanth, low-substituted hydroxypropyl cellulose, glucose, sorbitol.

### Fillers

A suitable filler may be selected from, for example, starch derivatives, such as corn starch, potato starch or rice starch, polysaccharides such as dextrins, maltodextrins, dextrates, microcrystalline cellulose, powdered cellulose, mixture of microcrystalline cellulose and guar gum, coprocessed blends of microcrystalline cellulose; and polyhydric alcohols, such as xylitol and sorbitol.

### Disintegrants

A disintegrant may be selected from, for example, alginic acid, carbon dioxide, carboxymethylcellulose calcium, carboxymethylcellulose sodium, microcrystalline cellulose, powdered cellulose, croscarmelose sodium, crospovidone, sodium docusate, gaur gum, hydroxypropyl cellulose, methylcellulose, polacrilin potassium, poloxamer, povidone, sodium alginate, sodium glycine carbonate, sodium lauryl sulfate, sodium starch glycolate, starch, pregelatinized starch, low-substituted hydroxypropyl cellulose.

### Glidants

A glidant may be selected from, for example, calcium silicate, powdered cellulose, starch, talc, colloidal silicon dioxide.

### Lubricants

A lubricant may be selected from, for example, magnesium stearate, stearic acid, sodium stearyl fumarate, magnesium lauryl sulphate, talc, polyethylene glycol, and glyceryl behenate, glyceryl monostearates, palmitic acid, talc, carnauba wax, calcium stearate sodium, sodium or magnesium lauryl sulfate, calcium soaps, zinc stearate, polyoxyethylene monostearates, calcium silicate, silicon dioxide, hydrogenated vegetable oils and fats, stearic acid, and any combinations thereof.

### Essential Oils

A suitable essential oil may be selected from Bergamot oil (extracted from Citrus aurantium L. subsp. bergamia Wright et Am.); Ylang ylang oil (extracted from Cananga odorata Hook. f. and Thoms.); Jasmine essential oil (extracted from Jasminum officinale L.). In one embodiment, a mixture of essential oils comprises equal portions totaling about 0.01% to about 1% w/w, preferably about 0.1% w/w of the total composition. Other essential oils are possible.

### Sweeteners/Flavoring Agents

A suitable sweetener may be selected from sugars such as sucrose, lactose and glucose; cyclamate and salts thereof; saccharin and salts thereof; and aspartame.

Flavoring agents may be incorporated in the composition may be chosen from synthetic flavors oils and flavoring aromatics, natural oils, plant extracts. Examples include cinnamon oil, oil of wintergreen, peppermint oil, clove oil, bay oil, anise oil, eucalyptus, thyme oil, cedar leaf oil, nutmeg oil, sage oil or almond oil. Examples of flavoring agents include, but are not limited to, almond, apple, banana, berry, bubblegum, caramel, citrus, cherry, chocolate, coconut, grape, green tea, honey, lemon, licorice, lime, mango, maple, mint, orange, peach, pineapple, raisin, strawberry, vanilla, watermelon and combinations thereof. Flavors may be present in an amount ranging from about 0.001001% to about 5% by total weight of the formulation. In some embodiments, the flavoring agent may be selected from natural or synthetic flavors such as, for example, strawberry flavor, wild cherry flavor, green apple flavor, spearmint flavor and peppermint flavor. In some embodiments, the flavoring agents are selected from menthol, peppermint, wintergreen, orange, cherry, and other fruits, vanilla, almond and other nuts, etc. In some embodiments, the DHH-B formulations include a mixture of two or more flavoring agents.

### Absorption Enhancers

Absorption enhancers for use in accordance with certain embodiments of the present disclosure include, for example, Gelucire 44/14; Gelucire 50/13; Tagat TO; Tween 80; isopropyl myristate, polysorbates, sorbitan esters, poloxamer block copolymers, PEG-35 castor oil, PEG-40 hydrogenated castor oil, caprylocaproyl macrogol-8 glycerides, PEG-8 caprylic/capric glycerides, sodium lauryl sulfate, dioctyl sulfosuccinate, polyethylene lauryl ether, ethoxydiglycol, propylene glycol mono-di-caprylate, glycerol monocaprylate, glyceryl fatty acids (C8-C18) ethoxylated, oleic acid, linoleic acid, glyceryl caprylate/caprate, glyceryl monooleate, glyceryl monolaurate, caprylic/capric triglycerides, ethoxylated nonylphenols, PEG-(8-50) stearates, olive oil PEG-6 esters, triolein PEG-6 esters, lecithin, d-alpha tocopheryl polyethylene glycol 1000 succinate, polycarbonate, sodium glycocholate, sodium taurocholate, cyclodextrins, citric acid, sodium citrate, triacetin, combinations thereof, and the like. In certain preferred embodiments, the absorption enhancer is triacetin.

### Solubilizers

The DHH-B formulations may include one or more solubilizers or complexing agents. In some embodiments, the solubilizer or complexing agent is a cyclodextrin. Cyclodextrins are cyclic oligosaccharides composed of cyclic α-(1→4) linked D-glucopyranose units. Cyclodextrins with six to eight units have been named α-, β- and γ-cyclodextrin, respectively. The number of units determines the size of the cone-shaped cavity which characterizes cyclodextrins and into which drugs may include to form stable complexes. A number of derivatives of α-, β- and γ-cyclodextrin are known in which one or more hydroxyl groups is/are replaced with ether groups or other radicals. These compounds are thus known complexing agents and have been previously used in the pharmaceutical field to form inclusion complexes with water-insoluble drugs and to thus solubilize them in aqueous media.

The cyclodextrins within the scope of the present disclosure include the natural cyclodextrins α, β, and γ-cyclodextrin, and derivatives thereof, in particular, derivatives wherein one or more of the hydroxy groups are substituted, for example, by alkyl, hydroxyalkyl, carboxyalkyl, alkylcarbonyl, carboxyalkoxyalkyl, alkylcarbonyloxyalkyl, alkoxycarbonylalkyl or hydroxy-(mono or polyalkoxy)alkyl groups; and wherein each alkyl or alkylene moiety preferably contains up to six carbons. Substituted cyclodextrins can generally be obtained in varying degrees of substitution, for example, from 1 to 14, preferably from 4 to 7; the degree of substitution is the approximate average number of substituent groups on the cyclodextrin molecule, for example, the approximate number of hydroxypropyl groups in the case of the hydroxypropyl-β-cyclodextrin molecule, and all such variations are within the ambit of this present disclosure. Substituted cyclodextrins which can be used in the present disclosure include polyethers, for example, as described in U.S. Pat. No. 3,459,731.

Further examples of substituted cyclodextrins include ethers wherein the hydrogen of one or more cyclodextrin hydroxy groups is replaced by C₁₋₆ alkyl, hydroxy-C₁₋₆ alkyl, carboxy-C₁₋₆ alkyl or C₁₋₆ alkyloxycarbonyl-C₁₋₆ alkyl groups or mixed ethers thereof. In particular, such substituted cyclodextrins are ethers wherein the hydrogen of one or more cyclodextrin hydroxy groups is replaced by C₁₋₃ alkyl, hydroxy-C₂₋₄ alkyl or carboxy-C₁₋₂ alkyl or more particularly by methyl, ethyl, hydroxyethyl, hydroxypropyl, hydroxybutyl, carboxymethyl or carboxyethyl. The term “C₁₋₆ alkyl” is meant to include straight and branched saturated hydrocarbon radicals, having from 1 to 6 carbon atoms such as methyl, ethyl, 1-methylethyl, 1,1-dimethylethyl, propyl, 2-methylpropyl, butyl, pentyl, hexyl and the like. Other cyclodextrins contemplated for use herein include glucosyl-β-cyclodextrin and maltosyl-β-cyclodextrin.

Of particular utility in the present disclosure are the β-cyclodextrin ethers such as dimethyl-β-cyclodextrin as described in Cyclodextrins of the Future, Vol. 9, No. 8, p. 577-578 by M. Nogradi (1984), randomly methylated β-cyclodextrin and polyethers such as hydroxypropyl-β-cyclodextrin, hydroxyethyl-β-cyclodextrin, hydroxypropyl-γ-cyclodextrin, and hydroxyethyl-γ-cyclodextrin, as well as sulfobutyl ethers, especially β-cyclodextrin sulfobutyl ether. In addition to simple cyclodextrins, branched cyclodextrins and cyclodextrin polymers may also be used. Other cyclodextrins are described, for example, in Chemical and Pharmaceutical Bulletin 28: 1552-1558 (1980); Yakugyo Jiho No. 6452 (28 Mar. 1983); Angew. Chem. Int. Ed. Engl. 19: 344-362 (1980); U.S. Pat. Nos. 3,459,731 and 4,535,152; European Patent Nos. EP 0 149 197A and EP 0 197 571A; PCT International Patent Publication No. WO90/12035; and UK Patent Publication GB 2,189,245.

Other references describing cyclodextrins for use in the compositions according to the present disclosure, and which provide a guide for the preparation, purification and analysis of cyclodextrins include the following: Cyclodextrin Technology by Jozsef Szejtli, Kluwer Academic Publishers (1988) in the chapter Cyclodextrins in Pharmaceuticals; Cyclodextrin Chemistry by M. L. Bender et al., Springer-Verlag, Berlin (1978); Advances in Carbohydrate Chemistry, Vol. 12, Ed. by M. L. Wolfrom, Academic Press, New York in the chapter “The Schardinger Dextrins” by Dexter French, pp. 189-260; Cyclodextrins and their Inclusion Complexes by J. Szejtli, Akademiai Kiado, Budapest, Hungary (1982); I. Tabushi, Acc. Chem. Research, 1982, 15, pp. 66-72; W. Sanger, Angewandte Chemie, 92, p. 343-361 (1981); A. P. Croft et. al., Tetrahedron, 39, pp. 1417-1474 (1983); Irie et. al., Pharmaceutical Research, 5, pp. 713-716 (1988); Pitha et. al., Int. J. Pharm. 29, 73 (1986); U.S. Pat. Nos. 4,659,696 and 4,383,992; German Patent Nos. DE 3,118,218 and DE-3,317,064; and European Patent No. EP 0 094 157A. Patents describing hydroxyalkylated derivative of (3- and γ-cyclodextrin include Pitha U.S. Pat. Nos. 4,596,795 and 4,727,064, Muller U.S. Pat. Nos. 4,764,604 4,870,060 and Muller et al. U.S. Pat. No. 6,407,079.

Cyclodextrins of particular interest for complexation with DHH-B include: γ-cyclodextrin; hydroxyalkyl, e.g. hydroxyethyl or hydroxypropyl, derivatives of β- and γ-cyclodextrin; carboxyalkyl, e.g. carboxymethyl or carboxyethyl, derivatives of β- or γ-cyclodextrin; β-cyclodextrin sulfobutyl ether; dimethyl-β-cyclodextrin; and randomly methylated β-cyclodextrin. 2-Hydroxypropyl-β-cyclodextrin (HPβCD), 2-hydroxypropyl-γ-cyclodextrin (HPγCD), randomly methylated β-cyclodextrin, dimethyl-β-cyclodextrin, β-cyclodextrin sulfobutyl ether, carboxymethyl-β-cyclodextrin (CMβCD), carboxymethyl-γ-cyclodextrin (CMγCD) and γ-cyclodextrin (γCD) itself are of special interest, especially γ-cyclodextrin and hydroxypropyl-, β-cyclodextrin, most especially γ-cyclodextrin.

### Surfactants

In some embodiments, the surfactant is an anionic surfactant. Anionic surfactants are generally based upon sulfates, sulfonates, phosphates, or carboxylates and contain a water-soluble cation. A representative formula of a sulfonate is R—SO₃—M, where R is a hydrocarbon group of from about 5 to 22 carbon atoms which may be linked through an alkoxy or oxyalkoxy to the sulfonate functionality, and where M is a water-soluble cation such as an alkali metal. In some embodiments, anionic surfactants include alkyl ether sulfates, alkyl sulfates and sulfonates, alkyl carboxylates, alkyl phenyl ether sulfates, sodium salts of alkyl poly(oxyethylene) sulfonates, sodium salts of alkyl benzyl sulfonate, such as sodium salts of dodecylbenzyl sulfonate and sodium lauryl ether sulfate. In some embodiments, anionic surfactants also include anionic phosphate esters.

In some embodiments, the anionic surfactants include, but are not limited to polyoxyethylene alkyl ether, wherein the alkyl is (CH₂)_{S} and the oxyethylene is (C₂H₄O)_{T}, wherein S is an integer from 5 to 16, from 8 to 14, or from 10 to 12; and T is an integer from 10 to 40, from 15 to 30, or from 20 to 28. In one embodiment, the anionic surfactant is polyoxyethylene lauryl ether having a formula (C₂H₄₀)₂₃C₁₂H₂₅OH. In another embodiment, the anionic surfactant is a polyoxyethylene (20) sorbitan monoalkylate, the monoalkylate comprising between 8 and 14 carbons. In another embodiment, the anionic surfactant is a linear secondary alcohol polyoxyethylene having a formula C₁₂₋₁₄H₂₅₋₂₉O(CH₂CH₂O]ₓ, wherein x is an integer ranging from between 2 and 12. In yet another embodiment, the anionic surfactant is a polyoxyethylene octyl phenyl ether. Exemplary surfactants are sold under the names: Brij® 35, TWEEN®, Tergitol™, Triton™, Ecosurf™, Dowfax™, polysorbate 80™, BigCHAP, Deoxy BigCHAP, IGEPAL®, Saponin, Thesit®, Nonidet®, Pluronic F-68, digitonin, deoxycholate, and the like. In some embodiments, the anionic surfactant is selected from Brij® 35, TWEEN®, Tergitol™, Triton™.

In some embodiments, the surfactant is a cationic surfactant. Cationic surfactants useful in formulations of the present disclosure include amino or quaternary ammonium moieties. Cationic surfactants among those useful herein are disclosed in the following documents: M.C. Publishing Co., McCutcheon's, Detergents & Emulsifiers, (North American edition 1979); Schwartz, et al.; Surface Active Agents, Their Chemistry and Technology, New York: Interscience Publishers, 1949; U.S. Pat. No. 3,155,591, Hilfer, issued Nov. 3, 1964; U.S. Pat. No. 3,929,678, Laughlin et al., issued Dec. 30, 1975; U.S. Pat. No. 3,959,461, Bailey et al., issued May 25, 1976; and U.S. Pat. No. 4,387,090, Bolich, Jr., issued Jun. 7, 1983.

In some embodiments, the quaternary ammonium-containing cationic surfactant materials useful herein are those of the general formula:

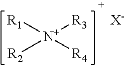

wherein R₁-R₄ are each independently an aliphatic group of from about 1 to about 22 carbon atoms or an aromatic, alkoxy, polyoxyalkylene, alkylamido, hydroxyalkyl, aryl or alkylaryl group having from about 1 to about 22 carbon atoms; and X is a salt-forming anion such as those selected from halogen, (e.g. chloride, bromide), acetate, citrate, lactate, glycolate, phosphate nitrate, sulfate, and alkylsulfate radicals. In some embodiments, the aliphatic groups may include, in addition to carbon and hydrogen atoms, ether linkages, and other groups such as amino groups. In some embodiments, the longer chain aliphatic groups, e.g., those of about 12 carbons, or higher, can be saturated or unsaturated. In some embodiments, the cationic surfactants are mono-long chain (e.g., mono C₁₂ to C₂₂, or C₁₂ to C₁₈) di-short chain (e.g., C₁ to C₃ alkyl) quaternary ammonium salts.

In some embodiments, the salts of primary, secondary and tertiary fatty amines are also suitable cationic surfactant materials. In some embodiments, the alkyl groups of such amines have from about 12 to about 22 carbon atoms and may be substituted or unsubstituted. Such amines include, but are not limited to, stearamido propyl dimethyl amine, diethyl amino ethyl stearamide, dimethyl stearamine, dimethyl soyamine, soyamine, myristyl amine, tridecyl amine, ethyl stearylamine, N-tallowpropane diamine, ethoxylated (with 5 moles of ethylene oxide) stearylamine, dihydroxy ethyl stearylamine, and arachidylbehenylamine. Suitable amine salts include the halogen, acetate, phosphate, nitrate, citrate, lactate, and alkyl sulfate salts. Such salts include, but are not limited to, stearylamine hydrochloride, soyamine chloride, stearylamine formate, N-tallowpropane diamine dichloride, stearamidopropyl dimethylamine citrate, cetyl trimethyl ammonium chloride and dicetyl diammonium chloride. In some embodiments, the cationic surfactant is a cetyl trimethyl ammonium chloride, stearyl trimethyl ammonium chloride, tetradecyltrimethyl ammonium chloride, dicetyldimethyl ammonium chloride, dicocodimethyl ammonium chloride and mixtures thereof. In other embodiments, the cationic surfactant is a cetyl trimethyl ammonium chloride.

In some embodiments, the surfactant is a non-ionic surfactant. Among the suitable nonionic surfactants are condensation products of C₈-C₃₀ alcohols with sugar or starch polymers. These compounds can be represented by the formula (S)ₙ—O—R, wherein S is a sugar moiety such as glucose, fructose, mannose, and galactose; n is an integer of from about 1 to about 1000, and R is C₈-C₃₀ alkyl. Examples of suitable C₈-C₃₀ alcohols from which the R group may be derived include decyl alcohol, cetyl alcohol, stearyl alcohol, lauryl alcohol, myristyl alcohol, oleyl alcohol, and the like. Suitable examples of such surfactants include decyl polyglucoside and lauryl polyglucoside.

Other suitable nonionic surfactants include the condensation products of alkylene oxides with fatty acids (i.e., alkylene oxide esters of fatty acids). These materials have the general formula RCO(X)ₙOH, wherein R is a C₁₀-C₃₀ alkyl, X is —OCH₂CH₂— (derived from ethylene oxide) or —OCH₂CHCH₃— (derived from propylene oxide), where n is an integer from about 1 to about 200.

Yet other suitable nonionic surfactants are the condensation products of alkylene oxides with fatty acids (i.e., alkylene oxide diesters of fatty acids) having the formula RCO(X)ₙOOCR, wherein R is a C₁₀-C₃₀ alkyl, X is —OCH₂CH₂— (derived from ethylene oxide) or —OCH₂CHCH₃— (derived from propylene oxide), where n is an integer from about 1 to about 200. Yet other nonionic surfactants are the condensation products of alkylene oxides with fatty alcohols (i.e., alkylene oxide ethers of fatty alcohols) having the general formula R(X)ₙOR′, wherein R is C₁₀-C₃₀ alkyl, where n is an integer from about 1 to about 200, and R′ is H or a C₁₀-C30 alkyl.

Yet further nonionic surfactants are compounds having the formula RCO(X)ₙOR′ where R and R′ are C₁₀-C30 alkyl, X is —OCH₂CH₂— (derived from ethylene oxide) or OCH₂CHCH₃— (derived from propylene oxide), and n is an integer from about 1 to about 200.

Examples of alkylene oxide-derived nonionic surfactants include ceteth-1, ceteth-2, ceteth-6, ceteth-10, ceteth-12, ceteraeth-2, ceteareth6, ceteareth-10, ceteareth-12, steareth-1, steareth-2, stearteth-6, steareth-10, steareth-12, PEG-2 stearate, PEG4 stearate, PEG6 stearate, PEG-10 stearate, PEG-12 stearate, PEG-20 glyceryl stearate, PEG-80 glyceryl tallowate, PPG-10 glyceryl stearate, PEG-30 glyceryl cocoate, PEG-80 glyceryl cocoate, PEG-200 glyceryl tallowate, PEG-8 dilaurate, PEG-10 distearate, and mixtures thereof. Still other useful nonionic surfactants include polyhydroxy fatty acid amides disclosed, for example, in U.S. Pat. Nos. 2,965,576, 2,703,798, and 1,985,424, which are incorporated herein by reference.

Non-limiting examples of surfactants include Tomadol 1200 (Air Products), Tomadol 900 (Air Products), Tomadol 91-8 (Air Products), Tomadol 1-9 (Air Products), Tergitol 15-S-9 (Sigma), Tergitol 15-S-12 (Sigma), Masurf NRW-N (Pilot Chemical), Bio-Soft N91-6 (Stepan), and Brij-35 (Polyethylene glycol dodecyl ether) (Sigma).

In some embodiments, the surfactant is selected from Polyhydroxyethyl alkoxy alkylene oxides, Polyoxyethylene-polyoxyprolyene block co-polymers, Etherified polyoxyethylene-polyoxyprolyene block co-polymers, Modified alkylated polyols, Modified/Methyl capped block co-polymers, Non-Ionic polyols, Non-ionic surfactants, Alkoxylated polyols., Alkyl polyglycosides, Glucoethers, Alkoxylated alcohols, Alcohol ethoxylates, Polyoxytheylene, Anioinic blends, Ethylene oxides, Nonylphenol ethoxylates, Sodium laureth sulfates, Laureth sulfates, Ammonium laureth sulfates, TEA lauryl sulfate, Diethylhexyl sodium sulfosuccinate, Sodium lauroyl sarcosinates, Sodium stearate, Sodium olefin sulfonate, Disodium laureth sulfosuccinate, Disodium oleamine sulfosuccinate, Sodium dioctyl sulfosuccinate Sodium cocoyl isethionate, Sodium capryloyl isethionate, Sodium caproyl isethionate, Sodium lauroyl isethionate, Sodium palmitoyl isethionate, Acrylates/Steareth-20 itaconate copolymer, Ammonium capryleth sulfate, Ammonium pareth-25 sulfate, Ammonium myreth sulfate, Ceteareth-20, Cocamidopropyl betaines, Disteareth-75 IPDI, -100 IPDI, Emulsifying wax NF, Isosteareth-20, Steareth-2, -4, 10, 16, -20, 21, Isosteareth-2, -10, -20, Magnesium laureth sulfate, Magnesium oleth sulfate, Polyethylene glycols, PEG-20, PEG-40, Phenoxyethanol, olyoxyethylene, Polysorbate-20, -40, -60, -80, Steareth-2, -4, -10, -16, -20, -21, Sodium coceth sulfate, Sodium deceth sulfate, Sodium oleth sulfate, Sodium laureth sulfate, Sodium syreth sulfate, Sodium trideceth sulfate, Zinc coceth sulfate, 2-Dodecylbenzenesulfonic acid, 4-Dodecylbenzenesulfonic acid, Alkylbenzene sulfonates, Glucoheptonates, odium glucoheptonate, Potassium glucoheptonate, Calcium glucoheptonate, Magnesium glucoheptonate, Boron glucoheptonate, Chlorine glucoheptonate, Copper glucoheptonate, Iron glucoheptonate, Manganese glucoheptonate, Molybdenum glucoheptonate, Zinc glucoheptonate, Methanoic acid, Ethanoic acid, Propanoic acid, Butanoic acid, Pentanoic acid, Hexanoic acid, Heptanoic acid, Octanoic acid, Nonanoic acid, Decanoic acid, Undecanoic acid, Dodecanoic acid, Tridecanoic acid, Tetradecanoic acid, Pentadecanoic acid, Hexadecanoic acid, Heptadecanoic acid, Octadecanoic acid, Nonadecanoic acid, Icosanoic acid, 1,2,3-trihydroxypropane, Diethylene glycol, Alkylphenol ethoxylate, 3 -oxapentane-1,5-diol, Propane-1,2,3-triol, Alkylphenol ethoxylate, Polydimethylsiloxane,1,2-Propanediol, Dimethylpolysiloxane, Fatty alcohol and butoxyethanol, Butoxyethanol, Phosphate ester surfactant, Alkyl aryl alkoxylate, Hydroxy carboxylic acids, Citric acid, Tartaric acid, Gluconic acid, Oxalic acid, Propionic acids, Phosphate ester, Ammonium sulfates, Ethoxylated surfactants, Sodium hydroxide, Anticorrosion compounds, Sequestering agents, Nonionic and Ionic surfactants, Hydroxy carbroxylates, Polyacrylates, Sugar acrylates, Aminocarboxylic acid base, Phosphate(s), Phosphonate(s), Sodium hexameta phosphate, sodium polyphosphate, Sodium tripolyphosphate, Sodium trimeta phosphate, Sodium pyrophosphates, Phosphonated aminopolycarboxylates, Amino polycarboxylates, EDTMP, DETMP, ATMP, HEDP, DTPMP, Polyether-polymethylsiloxane copolymer(s), Ethoxylated alkyl phosphate esters, C16-C28 alkanoates, Paraffin base petroleum oil, Agricultural paraffinic oil, Alkanolamide surfactants, Alkylaryl polyethoxyethanol sulfates, Alkylaryl polyoxyethylene glycol phosphate ester surfactants, Phosphate ester surfactants, Petroleum oil, Polyol fatty acid ester, Methylated seed oil, Paraffinic oil, Carbonyldiamide polyoxyalkylated glycol adduct, Carbonyl diamine, Polyoxyethylene-polyoxypropylene polymer, Methylated vegetable oil, Corn-derived surfactants, Free fatty acids, Isoproponal, Alkyl aryl polyoxyethylene glycols, Hydrogen sulfate, Aliphatic hydrocarbon oils, Polyacrylic acid salts, Polysiloxane polyether copolymer, Polyalkyleneoxide modified polydimethylsiloxane, Tall oil fatty acids, Organo silicone surfactant, Polyalkylene modified heptamethyltrisiloxane, Modified alkanoates, Poly fatty acid esters, Carbonate salts, Polysiloxane, Limonene, Allyloxypolyethyleneglycol methyl ether, Phytobland base oil, Dimethylpolysiloxane, Mineral oil, Polyether polymethylsiloxane copolymer, Nonionic carbohydrate surfactants, Polyoxythylenepolyoxyethylene-polyoxypropylene glycol, Monocarbamide dihydrogen sulfate, Antifoaming agents, Crop-based elasto polymer, Diammonium salts, and any combination thereof.

In some embodiments, the surfactant is a polyhydroxyethyl alkoxy alkylene oxide. In some embodiments, the surfactant is a polyoxyethylene-polyoxyprolyene block copolymer. In some embodiments, the surfactant is an etherified polyoxyethylene-polyoxyprolyene block copolymer. In some embodiments, the surfactant is a modified alkylated polyol. In some embodiments, the surfactant is a modified/methyl capped block copolymer. In some embodiments, the surfactant is a non-ionic polyol. In some embodiments, the surfactant is an alkoxylated polyol. In some embodiments, the surfactant is an alkyl polyglycoside. In some embodiments, the surfactant is a glucoether. In some embodiments, the surfactant is an alkoxylated alcohol. In some embodiments, the surfactant is an alcohol ethoxylate. In some embodiments, the surfactant is a polyoxytheylene. In some embodiments, the surfactant is an anioinic blend.

### Routes of Administration and Dosage Forms

Administration to a subject of the formulations according to the present disclosure may be via any common route so long as the target tissue is available via that route. The formulations may conveniently be presented in dosage unit form and may be prepared by any of the methods well known in the art of pharmacy. In some embodiments, the formulations are prepared by uniformly and intimately bringing the active components into association with a liquid carrier or a finely divided solid carrier or both, and then, if necessary, shaping the product into the desired dosage form. Of course, the skilled artisan will recognize that the active components (e.g. DHH-B) are included in an amount sufficient to produce the desired pharmacologic effect.

In some embodiments, the DHH-B formulations of the present disclosure may be provided, in the form of discrete units such as hard or soft capsules, tablets, troches or lozenges, each containing a predetermined amount of the active components; in the form of a dispersible powder or granules; in the form of a solution or a suspension in an aqueous liquid or non-aqueous liquid; in the form of syrups or elixirs; or in the form of an oil-in-water emulsion or a water-in-oil emulsion. In other embodiments, the DHH-B formulations of the present disclosure can be prepared in the form of, for example, a pharmaceutical preparation for oral administration or parenteral administration (e.g., intravenous, intraarterial, intraperitoneal, transrectal, subcutaneous, intramuscular, sublingual, intranasal, or transvaginal administration). Non-limiting examples thereof include solutions, tablets, powders, granules, capsules, suppositories, sprays, controlled-release agents, suspensions, and drinks.

By way of example, in solid dosage forms, the formulations may be mixed with at least one pharmaceutically acceptable excipient or carrier such as sodium citrate or dicalcium phosphate and/or a) fillers or extenders such as starches, lactose, sucrose, glucose, mannitol, dibasic calcium and silicic acid, b) binders such as, for example, microcrystalline cellulose, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, crospovidone, and sodium carbonate, e) solution retarding agents such as paraffin, f) absorption accelerators such as quaternary ammonium compounds, g) wetting agents such as, for example, acetyl alcohol and glycerol monostearate, h) absorbents such as kaolin and bentonite clay, i) lubricants such as talc, calcium stearate, magnesium stearate, solid polyethylene glycols, sodium lauryl sulfate, and j) solubilizers or complexing agents such as, for example, cyclodextrins, and mixtures thereof.

### Oral Dosage Forms

The DHH-B formulations may be provided, in general, in the form of discrete units such as hard or soft capsules, tablets, troches or lozenges, each containing a predetermined amount of DHH-B, such as described herein; in the form of a dispersible powder or granules; in the form of a solution or a suspension in an aqueous liquid or non-aqueous liquid; in the form of syrups or elixirs; or in the form of an oil-in-water emulsion or a water-in-oil emulsion.

In some embodiments, DHH-B liquid dosage forms for oral administration include, but are not limited to, pharmaceutically acceptable emulsions, solutions, suspensions, syrups and elixirs. In addition to DHH-B, any liquid dosage forms may contain inert diluents commonly used in the art. For instance, liquid DHH-B formulations may include water, alcohol, polyethylene glycol ethers, or any other pharmaceutically acceptable solvents. Solubilizing agents (e.g. cyclodextrins) and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof may also be present in the disclosed DHH-B formulations. Additionally, oral DHH-B formulations can include adjuvants such as wetting agents, emulsifying and suspending agents, sweetening, flavoring, and perfuming agents. When formulated as a suspension, the disclosed DHH-B formulations include the DHH-B active agent and suspending agents, for example, ethoxylated isostearyl alcohols, polyoxyethylene sorbitol, sorbitan esters, microcrystalline cellulose, aluminum metahydroxide, bentonite, agar-agar, tragacanth, and mixtures thereof.

In some embodiments, aqueous suspensions include the DHH-B in admixture with excipients suitable for the manufacture of aqueous suspensions. Such excipients may be (a) suspending agents such as hydroxy ethylcellulose, sodium carboxymethylcellulose, methylcellulose, hydroxypropylmethylcellulose, sodium alginate, polyvinylpyrrolidone, gum tragacanth and gum acacia; (b) dispersing or wetting agents which may be (b.1) a naturally-occurring phosphatide such as lecithin, (b.2) a condensation product of an alkylene oxide with a fatty acid, for example, polyoxyethylene stearate, (b.3) a condensation product of ethylene oxide with a long chain aliphatic alcohol, for example heptadecaethyleneoxycetanol, (b.4) a condensation product of ethylene oxide with a partial ester derived from a fatty acid and a hexitol such as polyoxyethylene sorbitol monooleate, or (b.5) a condensation product of ethylene oxide with a partial ester derived from a fatty acid and a hexitol anhydride, for example polyoxyethylene sorbitan monooleate.

In some embodiments, the DHH-B aqueous suspensions may also include one or more preservatives, for example, ethyl or n-propyl p-hydroxybenzoate; one or more coloring agents; one or more flavoring agents; and one or more sweetening agents, such as sucrose or saccharin.

In some embodiments, DHH-B oily suspensions may be formulated by suspending the DHH-B in a vegetable oil, for example arachis oil, olive oil, sesame oil or coconut oil, or in a mineral oil such as liquid paraffin. In some embodiments, the oily suspensions may include a thickening agent, for example beeswax, hard paraffin or cetyl alcohol. In some embodiments, sweetening agents and flavoring agents may be added to provide a palatable oral preparation.

In some embodiments, the DHH-B formulations may include at least one antioxidant, for enhancing the stability of a drug. The antioxidant may be present either as a part of a formulation or as a packaging component. Antioxidants can be present in amounts effective to retard decomposition of a drug that is susceptible to oxidation. In some embodiments, the content of an antioxidant in the DHH-B formulations ranges from about 0.001 to 10 by total weight of the DHH-B formulations. Non-limiting examples of antioxidants include one or more of ascorbic acid and its salts, tocopherols, sulfite salts such as sodium metabisulfite or sodium sulfite, sodium sulfide, butylated hydroxyanisole, butylated hydroxytoluene, ascorbyl palmitate, and propyl gallate. Other suitable antioxidants will be readily recognized by those skilled in the art.

In some embodiments, the DHH-B formulations may include an emulsifier. The emulsifier may be any known to those of ordinary skill in the art. In some embodiments, the emulsifier is a block copolymer containing a polyoxyethylene block, i.e. a block made up of repeating ethylene oxide moieties. A suitable emulsifier of this type is Poloxamer, i. e. a polyoxyethylene-polyoxypropylene block copolymer, such as Poloxamer 188. See the Handbook of Pharmaceutical Excipients, p. 352, 2nd Edn. Pharmaceutical Press, London, 1994, Eds, Wade and Weller.

In some embodiments, the emulsifier is a phospho emulsifier. This can be any pharmaceutically acceptable material derived from soybeans or eggs, e.g. soy or egg lecithins. Egg lecithins, such as the material provided by Lipoid (Germany) known as Lipoid E80, which contains both phosphatidylcholine and phosphatidyl ethanoline, are preferred, although other phospholipid materials could be used including phospholipid- polyethylene glycol (PEG) conjugates (PEGylated phospholipids) that have been described for use in liposome systems, e. g. by Litzinger et al, Biochem Biophys Acta, 1190 (1994) 99-107.

Exemplary phospholipids suitable for oral dosage forms include: Phosal® 50 PG; Phosal® 53MCT; Phosal® 75SA, Phospholipon® 80; Phospholipon®80H; Phospholipon®85G; Phospholipon® 90G; Phospholipon® 90H; and Phospholipon® 90NG. Exemplary phospholipids suitable for dermal dosage forms include: Phosal® 50 PG; Phosal® 50SA; Phosal®53MCT; Phosal® 75SA; Phospholipon® 80; Phospholipon® 80H; Phospholipon® 85G; Phospholipon® 90NG; Phospholipon®90G; Phospholipon® 90H; and Phospholipon® 100H. Exemplary phospholipids suitable for parenteral dosage forms include: Phospholipon®90G; Phospholipon®90H; and Phospholipon® 100H. Phosholipids suitable for pulmonary drug formulations include: Phospholipon® 90G; Phospholipon® 9.0H and Phospholipon®.

In some embodiments, the emulsifier may comprise a polysaccharide. The polysaccharide may be linear or branched, sulfated or unsulfated. In some embodiments, the composition comprises one or more linear sulfated polysaccharide known as “carrageenan”.

In some embodiments, the emulsifier is a carrageenan, for example one or more of a lambda-carrageenan, kappa-carrageenan, iota-carrageenan and any mixture of the carrageenans. The carrageenans are a family of linear sulfated polysaccharides that are extracted from edible seaweed and widely used in the food industry. The USPNF 23 describes carrageenan as hydrocolloid obtained by extraction and purification with water or aqueous alkali from few members of the class Rhodophyceae (red seaweed). It consists mainly of potassium, sodium, calcium magnesium and ammonium sulfate esters of galactose and 3,6-anhydrogalactose copolymers. These hexoses are alternatively linked at the α-1,3 and β-1,4 sites in the polymer.

Carrageenans are divided into three families according to the position of sulfate groups and the presence of anhydrogalactose. Lambda-carrageenan (λ-carrageenan) is a nongelling polymer containing about 35% ester sulfate by weight and no 3,6-anhydrogalactose. Iota-carrageenan (t-carrageenan) is a gelling polymer containing about 32% ester sulfate by weight and approximately 30% 3,6-anhydrogalactose. Kappa carrageenan (κ-carrageenan) is a strongly gelling polymer which has a helical tertiary structure that allows gelling. It contains 25% ester sulfate by weight and approximately 34% 3,6-anhydrogalactose. Among the three carrageenans, λ-carrageenan is the only nongelling polymer.

In some embodiments, the emulsifier is selected from the group consisting of a monoglyceride, a diglyceride, and any mixture thereof. The term “monoglyceride” refers to a molecule with one glycerol moiety covalently bonded to a fatty acid chain via an ester bond. The term “diglyceride” refers to a molecule with one glycerol moiety covalently bonded to two fatty acid chains via ester bonds. In some embodiments, the emulsifier includes a mixture of monoglycerides and diglycerides. In some embodiments, the emulsifier includes a mixture of monoglycerides and diglycerides and a carrageenan.

In some embodiments, the emulsifier is a lecithin (which is a generic term to designate any group of fatty substances occurring in animal and plant tissues that are composed of phosphoric acid, choline, fatty acids, glycerol, glycolipids, triglycerides, and phospholipids (e.g., phosphatidylcholine, phosphatidylethanolamine, and phosphatidylinositol)). In some embodiments, triglycerides include vegetable oils, fish oils, animal fats, hydrogenated vegetable oils, partially hydrogenated vegetable oils, medium and long-chain triglycerides, and structured triglycerides. In some embodiments, the emulsifier may be an oil, including vegetable oils such as soybean oil, olive oil, cotton seed oil, peanut oil, sesame oil and castor oil, with sesame oil and castor oil being preferred.

In some embodiments, the emulsifier is a fatty acid ester of polyoxyethylene sorbitan (e.g. Polysorbate® 80, Polysorbate® 20, etc.).

In some embodiments, the DHH-B formulations may be provided in the form of dispersible powders and/or granules, such as those suitable for the preparation of an aqueous suspension. In some embodiments, DHH-B is provided in admixture with a dispersing or wetting agent, a suspending agent and one or more preservatives. Suitable dispersing or wetting agents and suspending agents are exemplified by those already described herein. Additional excipients, for example, those sweetening, flavoring and coloring agents described above may also be present, including each of those described herein. Coloring agents can be used to color code compositions, for example, to indicate the type and dosage of the therapeutic agent therein. Coloring agents can also be used to differentiate the varied fractions of multiparticulates comprised in a unit dosage form such as a capsule. Suitable coloring agents include, without limitation, one or more natural and/or artificial colorants such as FD&C coloring agents, natural juice concentrates, pigments such as titanium oxide, silicon dioxide, iron oxides, zinc oxide, and the like.

The DHH-B formulations of the present disclosure may also be in the form of oil-in-water emulsions. In some embodiments, the oily phase may be a vegetable oil such as olive oil or arachis oils, or a mineral oil such as liquid paraffin or a mixture thereof. Suitable emulsifying agents may be (a) naturally-occurring gums such as gum acacia and gum tragacanth, (b) naturally-occurring phosphatides such as soybean and lecithin, (c) esters or partial esters derived from fatty acids and hexitol anhydrides, for example, sorbitan monooleate, (d) condensation products of said partial esters with ethylene oxide, for example polyoxyethylene sorbitan monooleate. The emulsions may also contain sweetening and flavoring agents.

In some embodiments the DHH-B formulations may be provided in the form of syrups and elixirs may be formulated with sweetening agents, for example, glycerol, propylene glycol, sorbitol or sucrose. Such formulations may also contain a preservative and flavoring and coloring agents.

In some embodiments, solid dosage forms suitable for oral administration include, capsules, tablets, pills, powders, orally disintegrating tablets, and granules. In some embodiments, DHH-B solid dosage formulations may also be formulated into candies, lollipops, lozenges, etc. In such solid dosage forms, DHH-B may be mixed with at least one pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, acetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For capsules, tablets and pills, the dosage form can also comprise buffering agents.

An example of an orally dissolving tablet formulation is provided below:

| Component | Component | Range by total | Component |
|---|---|---|---|
| Name | amount | weight | Type |
| D-Mannitol | about 1 mg to | about 0.4% to | Diluent & |
| | about 162.5 mg | about 65% w/w | Palatability |
| Xylitol | about 1 mg to | about 0.4% to | Sweetener |
| | about 162.5 mg | about 65% w/w | |
| Microcrystalline | about 1 mg to | about 0.4% to | Binder |
| Cellulose | about 162.5 mg | about 65% w/w | |
| Crospovidone | about 1 mg to | about 0.4% to | Disintegrant |
| | about 162.5 mg | about 65% w/w | |
| Dibasic Calcium | about 1 mg to | about 0.4% to | Functional |
| Phosphate Anhydrous | about 162.5 mg | about 65% w/w | Filler |
| HPBCD Complexed | about 67.5 mg | about 20% to | Solubilizer |
| DHH-B | HPBCD and about | about 60% w/w | Complexed |
| | 7.5 mg DHH-B | | with Active |
| Magnesium Stearate | about 1.25 mg | about 0.25% to | Lubricant |
| 0.5% by weight | | about 1.0% w/w | |

An example of an oral tablet formulation (such as for a human subject) is provided below:

| Component | | Range by total | Component |
|---|---|---|---|
| Name | Component amount | weight | Type |
| HPBCD | about 67.5 mg | about 20% to | Solubilizer |
| Complexed | HPBCD and about | about 60% w/w | Complexed |
| DHH-B | 7.5 mg DHH-B | | with Active |
| Magnesium Stearate | about 1.25 mg | about 0.25% to | Lubricant |
| 0.5% by weight | | about 1.0% w/w | |
| Microcrystalline | about 162.5 mg | about 39% to | Binder |
| Cellulose | | about 79.75% w/w | |

Alternative lubricants include glyceryl tristearate, Nu-MAG®. Poem TR-FB (TR-FB) and Poem TR-HB (TR-HB). Alternative binders include Mannitol, Dextrose, Sucrose, Ethyl Cellulose, Methyl Cellulose, Hydroxy Propyl Methyl Cellulose, Sodium Carboxy Methyl Cellulose, Polyvinyl Pyrrolidone.

An example of an oral tablet formulation (such as for a canine subject) is provided below:

| Component | Component | Range by total | Component |
|---|---|---|---|
| Name | amount | weight | Type |
| DHH-B | about 12 mg | about 0.5 to | Active |
| | | about 1.0% w/w | |
| Dried Whey | about 1000 mg | about 70 to | Binder |
| | | about 80% w/w | |
| Microcrystalline | about 200 mg | about 10 to | Binder |
| Cellulose | | about 20% w/w | |
| Chicken or Liver | To formulation | about 0.1 to | Flavoring Agent |
| Flavoring | specifications | about 10% w/w | |
| Mg Stearate | about 6 mg | about 0.5% w/w | Lubricant |

In some embodiments, tablets may have a hardness ranging from about 20 Newtons to about 150 Newtons. In other embodiments, tablets may have a hardness ranging from about 20 Newtons to about 120 Newtons. In some embodiments, tablets may have a hardness ranging from about 20 Newtons to about 150 Newtons. In some embodiments, tablets may have a hardness ranging from about 20 Newtons to about 10 Newtons. In some embodiments, tablets may have a hardness ranging from about 20 Newtons to about 80 Newtons. In some embodiments, tablets may have a hardness ranging from about 20 Newtons to about 60 Newtons. In some embodiments, tablets may have a hardness ranging from about 20 Newtons to about 150 Newtons.

In some embodiments, DHH-B formulations for oral use may be in the form of hard gelatin or HPMC capsules wherein the active components are mixed with an inert solid diluent, for example pregelatinized starch, calcium carbonate, calcium phosphate or kaolin, or dispensed via a pellet formulation. In other embodiments, they may also be in the form of soft gelatin capsules wherein the active ingredient is mixed with water or an oil medium, for example peanut oil, liquid paraffin, medium chain triglycerides or olive oil.

In some embodiments, the tablets, capsules or pellets may be uncoated or they may be coated by known techniques to delay disintegration and absorption in the gastrointestinal tract and thereby provide a delayed action or sustained action over a longer period. For example, a time delay material such as celluloseacetate phtalate or hydroxypropylcellulose acetate succinate or sustained release material such as ethylcellulose or ammoniomethacrylate copolymer (type B) may be employed.

### Suppository Formulations

Suppository formulations within the scope of the present disclosure are prepared by admixing a therapeutically effective amount of DHH-B with a suppository base which provides long term stability to the suppository formulation and the forming of the suppositories from the admixture by any recognized method of making suppositories. Typically, such suppository bases are those which are lipophilic, more preferably, the suppository base is an aprotic lipophilic base such as a triglyceride lipophilic base or a paraffinic base comprising mixtures of hydrocarbons. The suppository base should have a melting temperature that ensures melting of the suppository within a reasonable time after insertion. Typically the suppository base can include mixtures of hydrocarbons (paraffins) having a melting point range of from about 32° C. to 36° C. or a triglyceride mixture of fatty acids having a melting point range of from about 32° C. to 36° C. The mixture of hydrocarbons can preferably be a mixture of hard paraffin (about 50-60%) and liquid paraffin (about 40-50%) having a melting point range of about 32 to 36° C. The preferred bases for suppositories are Witepsol S55, Witepsol S58, mixtures of these products, or mixtures of either, or both, with Witepsol W35 and/or Witepsol H15. The suppository base Witepsol is a mixture of mono-, di- and triglycerides which are glyceryl esters of plant derived fatty acid mixtures derived from palm seed oils, such as coconut oil.

In some embodiments, the suppository base has a conventional formulation and may contain cocoa butter, glycerin gelatin, hydrogenated vegetable oils, mixtures of polyethylene glycols of various molecular weights, polyethylene glycol fatty acid esters and mixtures of mono-, di-, and triglycerides which are glyceryl esters of mixtures of fatty acids of plant origin, such as derived from palm kernel oil (such as coconut oil and palm kernel oil) and less than 0.5% surfactant than Polysorbate 80 or Cetomacrogol 1000. The DHH-B suppository formulation of the present disclosure may also include other conventional ingredients, such as amine neutralizing agents, to provide a pH of from about 4 to about 8.5 in the suppository/water base emulsion. and to solubilize the hydrocolloid, for example polyacrylic acid.

Any glycerides, including triglycerides, diglycerides, and/or monoglycerides may be used in the DHH-B formulations of the present discslosure. In some embodiments, the triglyceride is one of LLL, OLL, OOL, OOO, PLL, POL, POO, or SOL. In one embodiment, triglycerides can also include SSL, SLS, LLS, LSL, MML, MLM, MML, LLM, SSM, SMS, MMM, SSS, and LLL. Long, medium, short, and mixed length chain triglycerides can be used. Triglycerides also include any triglyceride including residues of any known fatty acids, or any other shorter chain saturated or unsaturated acids. Fatty acids include myristoleic acid, palmitoleic acid, sapienic acid, oleic acid, elaidic acid, vaccenic acid, linoleic acid, linoelaidic acid, a-linolenic acid, arachidonic acid, eicosapentaenoic acid, erucic acid, docosahexaenoic acid, caprylic acid, capric acid, lauric acid, myristic acid, palmitic acid, stearic acid, arachidic acid, behenic acid, lignoceric acid, and/or cerotic acid. While fatty acids are primarily present in the formulations described herein as residues part of a triglyceride, diglyceride, or monoglyceride, independent fatty acids can be part of the formulations as well.

An example of a DHH-B suppository formulation is provided below:

| Component | Component | Range by total | Component |
|---|---|---|---|
| Name | amount | weight | Type |
| DHH-B | about 15 mg | about 0.75% to about | Active |
| | | 2.5 % w/w | |
| Mono, di-, and | about 110 mg | about 5% to about | Oil |
| triglycerides and PEG | | 50% w/w | |
| mono- and di-, esters | | | |
| of saturated fatty | | | |
| acids | | | |
| mono, di- and | about 800 mg | about 45% to about | Surfactant |
| triglycerides and | | 90% w/w | |
| mainly PEG-32 (MW | | | |
| 1500) mono- and | | | |
| diesters of lauric acid | | | |
| (C₁₂) | | | |
| Diethylene glycol | about 50 mg | about 0.5% to about | Cosurfactant |
| monoethyl ether | | 3% w/w | or solvent |

### Topical Administration

Dosage forms for topical administration include, but are not limited to, ointments, creams, emulsions, lotions, gels, sunscreens and agents that favor penetration within the epidermis. Various additives, known to those skilled in the art, may be included in the topical formulations of the present disclosure. Examples of additives include, but are not limited to, solubilizers, skin permeation enhancers, preservatives (e.g., anti-oxidants), moisturizers, gelling agents, buffering agents, surfactants, emulsifiers, emollients, thickening agents, stabilizers, humectants, dispersing agents and pharmaceutical carriers. Examples of moisturizers include jojoba oil and evening primrose oil.

Suitable skin permeation enhancers are well known in the art and include lower alkanols, such as methanol ethanol and 2-propanol; alkyl methyl sulfoxides such as dimethylsulfoxide (DMSO), decylmethylsulfoxide (C10 MSO) and tetradecylmethyl sulfoxide; pyrrolidones, urea; N,N-diethyl-m-toluamide; C₂-C₆ alkanediols; dimethyl formamide (DMF), N,N-dimethylacetamide (DMA) and tetrahydrofurfuryl alcohol. Other suitable penetration enhancers may include fatty acids such as oleic acid, lauric acid, capric acid, myristic acid, palmitic acid, stearic acid, linoleic acid, linolenic acid, dicaprate, reclineate, monoolein, dilaurin, caprylic acid, arachidonic acid, glyceryl 1-monocaprate, mono and di glycerides and physiologically acceptable salts thereof.

Examples of solubilizers include, but are not limited to, hydrophilic ethers such as diethylene glycol monoethyl ether (ethoxydiglycol, available commercially as Transcutol®) and diethylene glycol monoethyl ether oleate (available commercially as Softcutol®); polyoxy 35 castor oil, polyoxy 40 hydrogenated castor oil, polyethylene glycol (PEG), particularly low molecular weight PEGs, such as PEG 300 and PEG 400, and polyethylene glycol derivatives such as PEG-8 caprylic/capric glycerides (available commercially as Labrasol®); alkyl methyl sulfoxides, such as DMSO; pyrrolidones, DMA, and mixtures thereof.

In some embodiments, the DHH-B topical formulations may also include one or more wetting agents. Useful wetting agents include by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters or polysorbates (e.g., TWEEN®), polyethylene glycols, polyoxyethylene stearates, phosphates, sodium lauryl sulphate, poloxamer, sodium dodecylsulfate, carboxymethylcellulose calcium, carboxymethylcellulose sodium, methylcellulose, hydroxyethylcellulose, hydroxyl propylcellulose, hydroxypropylmethylcellulose phthalate, noncrystalline cellulose, magnesium aluminum silicate, triethanolamine, polyvinyl alcohol, and polyvinylpyrrolidone (or PVP). Tyloxapol (a nonionic liquid polymer of the alkyl aryl polyether alcohol type, also known as superinone or triton) is another useful wetting agent, combinations thereof and other such materials known to those of ordinary skill in the art.

An example of a DHH-B topical formulation is provided below:

| Component | Component | Range by total | Component |
|---|---|---|---|
| Name | amount | weight | Type |
| DHH-B | about 15 mg | about 0.000008 to | Active |
| | | about 1% w/w | |
| Propylene Glycol | about 885 mg | about 0.00005 to | Wetting |
| | | about 6% w/w | Agent |
| Phospholipid Base | about 49 mL | about 90-99.9% w/w | Carrier |
| (e.g. Lipoderm ® | | | |
| Transdermal Bases by | | | |
| PCCA, or PLO gel | | | |
| (Premium Lecithin | | | |
| Organogel)) | | | |

### Parenteral Formulations

DHH-B formulations for parenteral administration include aqueous and non-aqueous sterile injection solutions, which may contain anti-oxidants, buffers, bacteriostats and solutes which render the formulation isotonic with the blood of the intended recipient. Formulations for parenteral administration also include aqueous and non-aqueous sterile suspensions, which may include suspending agents and thickening agents. The formulations may be presented in unit-dose or multi-dose containers, for example sealed ampoules and vials, and may be stored in a freeze-dried (lyophilized) condition requiring only the addition of a sterile liquid carrier, for example saline, phosphate-buffered saline (PBS) or the like, immediately prior to use. Extemporaneous injection solutions and suspensions may be prepared from sterile powders of the kind described below.

An example of an injectable formulation is provided below:

1 Liter Formulation:

25,000 mg/L=25 mg/ml DHH-B

| | | | |
|---|---|---|---|
| HPBCD | about 225 g HPBCD and | about 5 to about | Solubilizer Complexed |
| Complexed DHH-B | about 25 g DHH-B | 30% w/w | with Active |
| Sterile Water for | about 700 to about | about 65 to about | Solvent |
| Injection | 950 ml | 90% w/w | |
| Benzyl Alcohol | about 50 ml | about 5% w/w | Preservative |

### Solid and Liquid Food Products

An effective amount of any one of the DHH-B formulations may be added to an arbitrary food or beverage product or functional food that does not substantially contain a dihydrohonokiol or a derivative or analog. Thus, of any one of the DHH-B formulations of the present disclosure may be prepared in the form of a food or beverage product or functional food. Examples of food or beverage products and functional foods include, but are not limited to, confectioneries, retort pouch food, juice, teas, and dairy products. In addition, sweetening agents, seasonings, emulsifiers, suspending agents, antiseptics, or the like can be added to the food or beverage product or functional food, according to need. Further, the anti-anxiety composition of the present disclosure can be used as a food additive.

A food product, dietary composition, or supplement according to the present disclosure is any ingestible preparation that contains the natural product compositions of the present disclosure mixed with a food product or dietary supplement composition. The food product can be dried, cooked, boiled, lyophilized or baked. A food composition or food product can comprise a bakery product, including but not limited to bread, pastries, brownies, cakes, pies, donuts, crackers, and muffins. A food composition or food product can comprise a dairy product, including but not limited to milk, fermented milk, curd, whey, yogurt, cream, cheese, butter, clarified butter, ghee, and ice cream. A food composition or food product can comprise a nut butter or seed butter, including but not limited to peanut butter, almond butter, cashew butter, hazelnut butter, macadamia nut butter, pecan butter, pistachio butter, walnut butter, pumpkin seed butter, sesame seed butter, soybean butter, and sunflower seed butter. A food composition or food product can comprise an oil (e.g., a cooking oil), including but not limited to olive oil, coconut oil, vegetable oil, canola oil, corn oil, peanut oil, sunflower seed oil, almond oil, avocado oil, rice bran oil, cottonseed oil, flaxseed oil, linseed oil, grape seed oil, hemp oil, mustard oil, macadamia oil, palm oil, tea seed oil, walnut oil, margarine, lard, butter, clarified butter, ghee, or tallow. A food composition or food product can comprise sports food products such as energy gels, sports drinks, energy powders, energy bars, energy shots, protein powders, and protein drinks (e.g., protein shakes). A food composition or food product can comprise a beverage, including but not limited to water, electrolyte drinks, soda, coconut water, tea (e.g., Jun tea, black tea, green tea, white tea, herbal tea), coffee, a soft drink, an alcoholic beverage (e.g., cocktail, liquor, spirits, beer, wine, malt beverage), water, juice (e.g., apple juice, orange juice, tomato juice, vegetable juice, cranberry juice), a sports drink, electrolyte-enriched water, vitamin-enhanced water, milk (e.g., dairy-based milk, coconut milk, almond milk, soy milk, hemp milk, rice milk, oat milk, cashew milk, hazelnut milk), and yogurt.

A food composition or food product may include the natural product composition disclosed herein within a gelatin-based product (e.g. Jell-O®) or gelatin-based desert. In some embodiments, the food composition or food product comprises the natural product composition and a gelling agent. Suitable examples of gelling agents include carrageenans, agar, sodium alginate, gellan gum, xanthan gum, sodium carboxymethyl cellulose, guar gum, soybean protein, and crystalline cellulose. The amount of the gelling agent contained within the food composition or food product is not limited provided that the effect of the present disclosure is obtained. The proportion of the gelling agent in the food composition is, for example, about 0.5 to about 3 by total weight of the food composition or food product. Without wishing to be bound by any particular theory, it is believed that the amount of the gelling agent contained affects the hardness in mastication. If the amount of gelling agent is less than about 0.5 by total weight of the food composition or food product, the food composition or food product tends to become overly soft and cannot achieve a hardness suitable for mastication. If, on the other hand, the amount is more than about 3 by total weight of the food composition or food product, the food composition or food products tends to fail to achieve a hardness at which chewing can be performed, even if the number of mastications is increased.

An example of a DHH-B liquid or beverage formulation is provided below:

| Component | Component | Range by total | Component |
|---|---|---|---|
| Name | amount | weight | Type |
| DHH-B | about 15 mg | about 0.000008 to | Active |
| | | about 1% w/w | |
| D-α-tocopheryl | about 90 mg | about 0.00005 to | Amphiphile |
| polyethylene glycol | | about 6% w/w | |
| succinate | | | |
| Water | about 50 mL | about 90 to about | Solvent |
| | | 99.9% w/w | |
| Flavoring | To formulation | about 0.1 to about | Taste Masking |
| | specifications | 10% w/w | Agent |

| Component | Component | Range by total | Component |
|---|---|---|---|
| Name | amount | weight | Type |
| DHH-B | about 15 mg | about 0.000008 to | Active |
| | | about 1% w/w | |
| D-α-tocopheryl | about 90 mg | about 0.00005 to | Amphiphile |
| polyethylene glycol | | about 6% w/w | |
| succinate | | | |
| Food product (e.g. | about 50 mL | about 90 to about | Solvent |
| soda, energy drink, | | 99.9% w/w | |
| tea, liquid shot | | | |
| beverage) | | | |
| Flavoring | To formulation | about 0.1 to about | Taste Masking |
| | specifications | 10% w/w | Agent |

### Dosing and Dosing Schedules

One of ordinary skill will appreciate that effective amounts of DHH-B in any of the formulations used in the methods of the present disclosure can be determined empirically. It will be understood that, when administered to a subject (human or veterinary), the total daily usage of the formulation of the present disclosure will be decided by the attending physician or other medical professional within the scope of sound medical judgment. The specific therapeutically effective dose level for any subject will depend upon a variety of factors: the type and degree of the response to be achieved; the activity of the specific composition employed; the age, body weight, general health, sex and diet of the patient; the duration of the treatment; severity of the patient; drugs used in combination or coincidental with the method of the present disclosure; and like factors well known in the medical arts.

According to need, such dose may be administered several separate times, such as 2 or 3 times. Also, any of the DHH-B formulations of the present disclosure may be administered to a patient in combination with another anti-anxiety agent.

Also, an effective amount any of the DHH-B formulations of the present disclosure may be added to any forms of feed for livestock animals (e.g., horses, cattle, or pigs) or pet animals (e.g., cats or dogs) that do not substantially contain a carotenoid. Thus, any of the DHH-B formulations of the present disclosure can be prepared in the form of feed. Anxiety disorders of animals can be prevented or treated via ingestion of such feed. Accordingly, such forms of feed are effective for animal breeding.

In some embodiments, any of the DHH-B formulations are administered once per day. In other embodiments, any of the DHH-B formulations are administered once twice per day. In other embodiments, any of the DHH-B formulations are administered at least three times per day. In some embodiments, any of the DHH-B formulations may be administered every 12 hours. In other embodiments, any of the DHH-B formulations may be administered every 8 hours. In yet other embodiments, any of the DHH-B formulations may be administered every 4 hours. In even further embodiments, any of the DHH-B formulations may be administered on an as-needed basis, but where the number of dosages in a 24-hour period does not exceed a predetermined number of doses or a predetermined amount of each active component. In some embodiments, any of the DHH-B formulations are administered with food. In other embodiments, any of the DHH-B formulations are administered while in a fasted state.

Any of the DHH-B formulations described herein can be provided in a unit dosage form. A unit dosage can be an hourly dosage. A unit dosage can be a daily dosage. A unit dosage can provide about 1/24, 1/12, ⅛, ⅙, ¼, ⅓, ½, or all of a daily dosage of one or more compositions for a subject in need thereof. As noted herein, a unit dosage can take the form of a tablet, gel, liquid, food product, food bar, container of liquid of defined volume, or other forms described herein, packaged for one-time consumption or administration.

### Methods of Treatment

In certain embodiments, the present disclosure relates to a method of decreasing anxiety that is a result of an anxiety disorder, such as panic disorder with or without agoraphobia, agoraphobia without history of panic disorder, animal and other phobias including social phobias, obsessive-compulsive disorder, stress disorders including post-traumatic and acute stress disorder, and generalized or substance-induced anxiety disorder; neuroses; convulsions; migraine; depressive or bipolar disorders, for example single-episode or recurrent major depressive disorder, dysthymic disorder, bipolar I and bipolar II manic disorders, and cyclothymic disorder; psychotic disorders including schizophrenia; neurodegeneration arising from cerebral ischemia; attention deficit hyperactivity disorder; Tourette's syndrome; speech disorders, including stuttering; and disorders of circadian rhythm, e.g. in subjects suffering from the effects of jet lag or shift work.

In some embodiments, any of the DHH-B formulations may be administered to a subject to treat anxiety and related disorders, including amelioration of anxiety and related symptoms. In some embodiments, the present disclosure relates to a method for the treatment and/or prevention of anxiety, comprising administering a therapeutically effective amount of DHH-B, alone or in combination with a pharmaceutically acceptable carrier or excipient. In some embodiments, the method comprises administering any of the DHH-B formulations described herein. In some embodiments, the method comprises administering DHH-B as denoted herein, together with a pharmaceutically acceptable excipient, carrier, and/or additive. In some embodiments, the method comprises administration of DHH-B or any of the DHH-B formulations disclosed herein embedded within a food composition or food product.

In some embodiments, any of the DHH-B formulations disclosed herein may be administered in a combination therapy, i.e., either simultaneously in single or separate dosage forms or in separate dosage forms within hours or days of each other. Examples of compounds/drugs used in such combination therapies for the treatment of anxiety include without limitation, Citalopram, Duloxetine, Escitalopram, Fluoxetine, Fluvoxamine, Paroxetine, Sertraline, Trazodone, Venlafaxine, Clomipramine, Desipramine, Doxepin, Imipramine, Isocarboxazid, Phenelzine, Selegiline, Tranylcypromine, Alprazolam, Chlordiazepoxide, Clonazepam, Diazepam, Lorazepam, Oxazepam, Divalproex, Gabapentin, Pregabalin, Atenolol, Nadolol, Propranolol, Molindone, Olanzapine, Quetiapine, and Risperidone. In some embodiments, the compositions disclosed herein may be administered in a combination therapy, i.e., either simultaneously in single or separate dosage forms or in separate dosage forms within hours or days of each other. Examples of compounds/drugs used in such combination therapies for the treatment of depression include without limitation, selective serotonin reuptake inhibitors (SSRIs), serotonin and norepinephrine reuptake inhibitors (SNRIs), tricyclic antidepressants (TCAs), tetracyclic antidepressants, dopamine reuptake blockers, 5-HT1A receptor antagonists, 5-HT2 receptor antagonists, 5-HT3 receptor antagonists, monoamine oxidase inhibitors (MAOIs), and noradrenergic antagonists. Specific examples of anti-depressants include, but are not limited to, desvenlafaxine, duloxetine, levomilnacipran, venlafaxine, amitriptyline, amoxapine, clomipramine, desipramine, doxepin, imipramine, nortriptyline, protriptyline, trimipramine, bupropion, maprotiline, vilazodone, nefazodone, trazodone, vortioxetine, isocarboxazid, phenelzine, selegiline, tranylcypromine, and mirtazapine.

In some embodiments, any of the DHH-B formulations described herein may be administered to a subject to treat trembling disorders, essential tremor disorder, Parkinsonian tremor, dystonic tremor, cerebellar tremor, psychogenic tremor, orthostatic tremor, physiologic tremor, and the like. In some embodiments, the present disclosure relates to a method for the treatment of trembling disorders, essential tremor disorder, Parkinsonian tremor, dystonic tremor, cerebellar tremor, psychogenic tremor, orthostatic tremor, physiologic tremor, and the like, comprising administering a therapeutically effective amount of DHH-B, alone or in combination with a pharmaceutically acceptable carrier or excipient. In some embodiments, the method comprises administering any of the DHH-B formulations described herein. In some embodiments, the method comprises administering DHH-B as denoted herein, together with a pharmaceutically acceptable excipient, carrier, and/or additive. In some embodiments, the method comprises administration of DHH-B or any of the DHH-B formulations disclosed herein embedded within a food composition or food product.

### Stability

In some embodiments, the individual components of any of the DHH-B formulations of the present disclosure do not degrade to an unacceptable extent such that the final product has a shelf-life of at least about 2 years. As previously mentioned, this means that the active components within the dosage form remains within about 90 to about 110% of its initial amount in the dosage form during the desired (e.g., labeled) shelf-life of the dosage form (e.g., a minimum of about 2 years after the date of manufacture of the dosage form). In some embodiments, the compositions described herein can have a shelf half-life of at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 240, 270, 300, 330, or 360 days. In some cases, the compositions described herein can have a shelf half-life of at least about 1, 2, 3, 4, or 5 years.

### EXAMPLES

### Example 1—Dihdrohonkiol-B-Cyclodextrin Complexation Process

Into a 250 ml beaker was added 90 ml deionized water. The beaker was placed on a stirring plate and heated to 70° C. A magnetic stir bar was placed into the beaker and set to a speed that created a vortex in the water. Into the water was added 45 g of Hydroxypropyl-beta cyclodextrin (HPBCD). The cyclodextrin rapidly went into solution. Into the solution of water, 5 g of Dihydrohonokiol-B that was slowly added.

### Example 2—Water-Soluble Tablet Formulations

For the manufacturing of a water-soluble tablet ingredient, the solution was transferred to a vacuum oven and heat applied along with vacuum overnight to remove the water from the dihydrohonokiol cyclodextrin complex. This material has been used in beverages, tablets, and Oral Dissolving Tablets (ODT).

### 250 mg Oral Dissolving Tablet Example

75 mg HPBCD complexed DHH-B

35 mg D-Mannitol

35 mg Xylitol

35 mg Microcrystaline Cellulose

35 mg Crospovidone

35 mg Dibasic Calcium Phosphate Anhydrous

### Example 3—Suppository Formulations

Base materials were melted at 50° C. to form a homogenous lipid blend. Dihydrohonokiol-B (15 mg) was dissolved in this mixture by mechanical mixing. The resultant molten SEDDS blend was poured into a suppository mold of 1 g capacity. The suppositories were allowed to set at room temperature for 5-10 min and further hardened for 30 min at 10 ° C. The final SEDDS product was assessed for its appearance, stability at room temperature, and ease of removal from the mold.

1,000 mg Suppository Example

15 mg DHH-B

110 mg Oil Blend

800 mg Peg-32 blend

50 mg Diethylene glycol Monoethyl Ether

### Example 4—Liquid or Beverage Formulations

Into a 250 ml glass beaker as added 9 g of D-α-tocopheryl polyethylene glycol succinate (TPGS). A stir bar was added to the beaker and it was placed on a heated stir plate set to 60° C. at a slow stirring speed. After the TPGS was liquefied by melting, 1.5 g of Dihydrohonokiol-B was added. After stirring for 10 minutes, 150 ml of 70° C. water was added and stirring speed increased. The solution was heated for an additional 15 minutes. The solution was allowed to sit overnight and 0.5% polysorbate 80 was added to clarify the solution if needed. The solution was further diluted for the appropriate formulation.

Examples of formulations include a 30 ml tincture bottle with a concentration of 7.5 mg DHH-B per ml to a 12 oz beverage with 15 mg per serving.

15 mg Liquid Example

15 mg DHH-B

90 mg TPGS

50 ml Water

100 mg Powdered Cherry Flavoring

### Example 5—Transdermal Formulations

Dihydrohonkiol-B and the wetting agent propylene glycol were first incorporated into the Phospholipid Base. The formulation was then mixed with an Electronic Mortar and Pestle (EMP) (Unguator® Technology, e/s model) for 3 minutes at a setting of 7, sheared twice using an ointment mill (Exakt Technologies, Inc., 50 model) (once at a setting of 2 and once at a setting of 1), and remixed with the EMP (1 minute at a setting of 5) to achieve content uniformity.

### Transdermal Example

15 mg DHH-B

885 mg Propylene Glycol

49 ml PLO-Gel

### Example 6—Injectable Formulations

Into a 1500 ml Erlenmeyer flask was added 450 ml deionized water. The beaker was placed on a stirring plate and heated to 70° C. A magnetic stir bar was placed into the beaker and set to a speed that created a vortex in the water. Into the water was added 225 g of Hydroxypropyl-beta cyclodextrin (HPBCD). The cyclodextrin rapidly went into solution. Into the solution of water, 25 g of Dihydrohonokiol-B that was slowly added. Upon cooling to room temperature, Benzyl alcohol is added at a quantity of less than 5.0%. The solution is adjusted to a pH of 7.10. The solution is brought to a quantity sufficient (qs) to bring it to 1000 ml. This solution is filtered through a sterile 0.22 um filter into individual multi-use sterile vials.

### Injectable Example

75 mg DHH-B

675 mg HPBCD

3 ml Sterile Water

0.15 ml Benzyl Alcohol

In this example, a 440 kg thoroughbred horse can be injected with 2 ml to 4 ml. A volume of 5 ml is appropriate for nervous horses. A quantity of 10 ml of 25 mg/ml did not elicit any adverse events.

### Example 7—Oral Tablet Formulation

250 mg Oral Tablet Example

75 mg HPBCD complexed DHH-B

1.25 mg Magnesium Stearate

162.5 mg Microcrystalline Cellulose

Although the present disclosure has been described with reference to a number of illustrative embodiments, it should be understood that numerous other modifications and embodiments can be devised by those skilled in the art that will fall within the spirit and scope of the principles of this disclosure. More particularly, reasonable variations and modifications are possible in the component parts and/or arrangements of the subject combination arrangement within the scope of the foregoing disclosure, the drawings, and the appended claims without departing from the spirit of the disclosure. In addition to variations and modifications in the component parts and/or arrangements, alternative uses will also be apparent to those skilled in the art.

### ADDITIONAL EMBODIMENTS

Additional Embodiment 1. A composition comprising dihydrohonokiol-B (“DHH-B”) and at least one pharmaceutically acceptable carrier, wherein the DHH-B is present in an amount ranging from between about 0.5% to about 25% by total weight of the composition.

Additional Embodiment 2. The composition of additional embodiment 1, wherein the DHH-B is complexed with a solubilizer.

Additional Embodiment 3. The composition of additional embodiment 2, wherein the solubilizer is a cyclodextrin.

Additional Embodiment 4. The composition of additional embodiment 3, wherein the cyclodextrin is hydroxypropyl-β-cyclodextrin.

Additional Embodiment 5. The composition of additional embodiment 4, further comprising at least three of a diluent, a binder, a sweetener, a disintegrant, a filler, and a lubricant.

Additional Embodiment 6. The composition of additional embodiment 4, further comprising crospovidone in an amount ranging from between about 0.4% to about 65% by total weight of the composition.

Additional Embodiment 7. The composition of additional embodiment 2, where the composition comprises microcrystalline cellulose in an amount ranging from between about 39% to about 80% by total weight of the composition.

Additional Embodiment 8. The composition of additional embodiment 1, wherein the DHH-B is present in an amount ranging from between about 0.75% to about 2.5% by total weight of the composition.

Additional Embodiment 9. The composition of additional embodiment 8, further comprising an oil and a surfactant.

Additional Embodiment 10. The composition of additional embodiment 9, wherein the oil comprises a triglyceride.

Additional Embodiment 11. The composition of additional embodiment 9, wherein the surfactant comprises polyethyleneglycol having an average molecular weight of about 1500 g/mol.

Additional Embodiment 12. The composition of additional embodiment 9, further comprising diethylene glycol monoethyl ether.

Additional Embodiment 13. A composition comprising dihydrohonokiol-B (“DHH-B”) and at least one pharmaceutically acceptable carrier, wherein the DHH-B is present in an amount ranging from between about 0.000001% to about 5% by total weight of the composition.

Additional Embodiment 14. The composition of additional embodiment 13, further comprising D-α-tocopheryl polyethylene glycol succinate.

Additional Embodiment 15. The composition of additional embodiment 14, further comprising a flavoring agent.

Additional Embodiment 16. The composition of additional embodiment 15, wherein the DHH-B is present in an amount ranging from between about 0.000008% to about 1% by total weight of the composition.

Additional Embodiment 17. The composition of additional embodiment 13, further comprising a wetting agent.

Additional Embodiment 18. The composition of additional embodiment 17, wherein the wetting agent is propylene glycol, and wherein the propylene glycol is present in an amount ranging from between about 0.0005% to about 6% by total weight of the composition.

Additional Embodiment 19. A method of treating anxiety, or the symptoms thereof, comprising administering to a subject in need thereof the composition of any one of additional embodiments 1-18.

Additional Embodiment 20. The method of additional embodiment 19, wherein the formulation is administered such that at least 5 mg of the DHH-B is administered to the subject per day.

Additional Embodiment 21. The method of additional embodiment 19, further comprising co-administering a second active pharmaceutical ingredient to the subject, wherein the second active pharmaceutical ingredient is an anxiolytic agent.

Additional Embodiment 22. A method of treating essential tremor disorder, Parkinsonian tremor, dystonic tremor, cerebellar tremor, psychogenic tremor, orthostatic tremor, physiologic tremor comprising administering to a subject in need thereof the composition of any one of additional embodiments 1-18.

Additional Embodiment 23. The method of additional embodiment 22, wherein the formulation is administered such that at least 5 mg of the DHH-B is administered to the subject per day.

1. A composition comprising dihydrohonokiol-B (“DHH-B”) and dried whey, wherein the DHH-B is present in an amount ranging from between about 0.5% to about 1% by total weight of the composition.

2. The composition of claim 1, wherein the dried whey is present in an amount ranging from between about 70% to about 80% by total weight of the composition.

3. The composition of claim 2, further comprising a binder, a lubricant, and a flavoring agent.

4. A composition comprising dihydrohonokiol-B (“DHH-B”) and a food product, wherein the DHH-B is present in an amount ranging from between about 0.000008% to about 1% by total weight of the composition.

5. The composition of claim 4, wherein the food product is dried whey.

6. The composition of claim 5, wherein the dried whey is present in an amount ranging from between about 70% to about 80% by total weight of the composition.

7. The composition of claim 4, wherein the food product is liquid.

8. The composition of claim 7, wherein the liquid is selected from the group consisting of soda, an energy drink, tea, and a liquid shot beverage.

9. The composition of claim 4, further comprising a flavoring agent.

10. An injectable formulation comprising dihydrohonokiol-B (“DHH-B”) complexed with a solubilizer, sterile water, and a preservative, wherein an amount of the DHH-B complexed with the solubilizer present in the composition ranges from between about 5% to about 30% by total weight of the formulation.

11. The injectable formulation of claim 10, wherein the preservative is benzyl alcohol.

12. The injectable formulation of claim 11, wherein the benzyl alcohol is present in an amount ranging from between about 3% to about 5% by total weight of the formulation.

13. The composition of claim 10, wherein the solubilizer is a cyclodextrin.

14. The composition of claim 13, wherein the cyclodextrin is hydroxypropyl-β-cyclodextrin.
